(19) United States
(12) Patent Application Publication
(10) Pub. No.: US 2003/0072723 A1
(43) Pub. Date: Apr. 17, 2003

(54) SUNSCREEN PREPARATIONS CONTAINING SURFACE-ACTIVE MONO-OR OLIGOGLYCERYL COMPOUNDS, WATER-SOLUBLE UV FILTER SUBSTANCES AND, IF DESIRED, INORGANIC MICROPIGMENTS

(76) Inventors: HEINRICH GERS-BARLAG, KUMMERFELD (DE); ANJA MULLER, RUMPEL (DE)

Correspondence Address: NORRIS MCLAUGHLIN & MARCUS, P.A., 220 EAST 42ND STREET-30TH FLOOR, NEW YORK, NY, 10017, US

(21) Appl. No.: 08/987,468

(22) Filed: Dec. 10, 1997

(30) Foreign Application Priority Data
Dec. 11, 1996 (DE) 196 51 478.9

(51) Int. Cl. A61K 7/42; A61K 7/44; A61K 6/00; A61K 7/00

(52) U.S. Cl. 424/59; 424/60; 424/401

(57) ABSTRACT

Sun protection active ingredient combinations comprising

(a) one or more UV filter substances which bear one or more sulphonic acid groups or sulphonate groups on their molecular backbone and

(b) one or more surface-active substances selected from the group of mono- or oligoglyceryl esters.

The present invention relates to cosmetic and dermatological sunscreen preparations, in particular cosmetic and dermatological sunscreen skincare preparations.

In a particularly preferred embodiment, the present invention relates to water-resistant sunscreen preparations.

The damaging effect of the ultraviolet part of solar radiation on the skin is generally known. Whereas rays having a wavelength of less than 290 nm (the so-called UVC region) are absorbed by the ozone layer in the earth's atmosphere, rays in the region between 290 nm and 320 nm, the so-called UVB region, cause erythema, simple sunburn or even burns of varying severity.

The narrower region around 308 nm is given as a maximum for the erythematous activity of sunlight.

Numerous compounds are known for providing protection against UVB radiation, said compounds usually being derivatives of 3-benzylidenecamphor, 4-aminobenzoic acid, cinnamic acid, salicylic acid, benzophenone and also 2-phenylbenzimidazole.

It is also important to have filter substances for the region between about 320 nm and about 400 nm, the so-called UVA region, because its rays too can cause damage. For example, it has been found that UVA radiation leads to damage of the elastic and collagenous fibres of the connective tissue, causing premature ageing of the skin, and that it is to be regarded as a cause of numerous phototoxic and photoallergic reactions. The damaging effect of UVB radiation can be reinforced by UVA radiation.

However, UV radiation can also lead to photochemical reactions, in which case the photochemical reaction products intervene in the skin's metabolism.

Such photochemical reaction products are predominantly free-radical compounds, for example hydroxyl radicals. Undefined free-radical photochemical products formed in the skin itself can also display uncontrolled subsequent reactions because of their high reactivity. However, singlet oxygen, a non-radical excited state of the oxygen molecule, can also occur under UV irradiation, as can short-lived epoxides and many other species. Singlet oxygen, for example, differs from the normal triplet oxygen (radical ground state) by its increased reactivity. However, excited, reactive (radical) triplet states of the oxygen molecule also exist.

Furthermore, UV radiation is a type of ionizing radiation. There is thus the risk that UV exposure may also create ionic species, which, in turn, are then capable of oxidative intervention in the biochemical processes.

2-Phenylbenzimidazole-5-sulphonic acid and its salts, in particular the sodium, potassium and TEA salts, obtainable for example under the name Eusolex® 232 from Merck AG, which has the following structural formula:

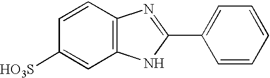

is a water-soluble UV filter substance which is advantageous per se.

UV absorbers or UV reflectors are mostly inorganic pigments, which are used in a known manner in cosmetics for protecting the skin from UV rays. Said inorganic pigments are oxides of titanium, zinc, iron, zirconium, silicon, manganese, aluminium and cerium and mixtures thereof, as well as modifications.

Inorganic pigments are notable per se for their good photoprotective effect. They do however have the disadvantage that it is difficult to incorporate them satisfactorily into such formulations. Only when the particles in the final formulation are very small are they not observed to produce a disturbing “whitening” (formation of white spots on the skin) following application to the skin. The particle sizes of such pigments are usually in the range below 100 nm. In a conventional emulsion the particles tend, to a greater or lesser extent, to form agglomerates which are visible even under the light microscope. Moreover, such agglomeration does not end with the manufacturing process of a particular preparation, but continues during storage. The “whitening” can therefore increase further over a prolonged period of time. In the medium or long term, this type of agglomeration can also lead to oil loss or even emulsion breakdown.

A further disadvantage of using inorganic pigments in cosmetic formulations is that such pigments lead to severe dryness of the skin in the vast majority of cases.

Nevertheless, the disadvantage of the prior art was that normally either only comparatively low sun protection factors could be achieved, or that the sunscreen filters had an insufficient UV stability or inadequate physiological compatibility or insufficiently high solubility or dispersibility in cosmetic or dermatological preparations, or exhibited other incompatibilities with cosmetic or dermatological preparations, or had several disadvantages at the same time.

Cosmetic or dermatological preparations are frequently in the form of finely disperse multiphase systems in which one or more fatty or oily phases are present alongside one or more aqueous phases. Of these systems, the actual emulsions are, in turn, the most widespread.

In simple emulsions, one phase contains finely disperse droplets of the second phase, surrounded by an emulsifier shell (water droplets in W/O emulsions or lipid vesicles in O/W emulsions). The droplet diameters of customary emulsions are in the range from approximately 1 μm to approximately 50 μm. Finer “macroemulsions”, whose droplet diameters are in the range from approximately 10⁻² μm to approximately 1 μm, are, again without colouring additives, bluish-white in colour and opaque.

The droplet diameter of transparent or translucent microemulsions, on the other hand, is in the range from approximately 10⁻² μm to approximately 10⁻¹ μm. Such microemulsions usually have a low viscosity. The viscosity of many microemulsions of the O/W type is comparable with that of water.

A disadvantage of many O/W emulsions of the prior art is that a high content of one or more emulsifiers must always be used, since the small droplet size results in a high interface between the phases, which as a rule must be stabilized by emulsifiers.

Water-soluble UV filter substances are electrolytes which destabilize, in particular, O/W emulsions. To counteract this destabilization, polyethoxylated emulsifiers are used. However, these often have dermatological disadvantages since, although the use of customary cosmetic emulsifiers is acceptable, emulsifiers, like any chemical substance, can nevertheless cause allergic reactions or reactions based on hypersensitivity of the user in individual cases.

For example, it is known that certain photodermatoses are triggered by certain emulsifiers, but also by various fats, and simultaneous exposure to sunlight. Such photodermatoses are also called “Mallorca acne”. An object of the present invention was therefore to develop sunscreen products.

Although there are completely advantageous cosmetic or dermatological preparations for protecting the skin from the harmful consequences of the effect of UV light, a disadvantage which is often observed is that the preparations are water-resistant to an inadequate extent if at all.

Sunscreen preparations are required and used particularly frequently on beaches and in open-air swimming pools. It is then desirable that the sunscreen formulation is largely water-resistant, that is to say that it is washed off from the skin only to a small extent or not at all.

Higher sun protection factors, that is to say, for example, those above SPF 15, can generally be achieved only by large amounts of UV filter substances. If a sun protection product is still to have a high sun protection factor even after bathing, the UV filter substance must, in particular, be retained on the skin.

It is already annoying if the sun protection product has to be applied again after bathing. During bathing itself, the use of a sunscreen formulation which can be washed off may even, under certain circumstances, be irresponsible and harmful to the skin, since water is a poor absorber of light in the UVA and UVB region, as a result of which it offers no notable UV protection, not even for submerged areas of skin.

For water-resistant sunscreen formulations, the prior art usually uses water-insoluble UV filter substances, water-repellent raw materials (e.g. silicone oils in high concentrations) and/or film formers, in particular high molecular weight compounds (e.g. PVP-hexadecene copolymers). Barriers are formed between the UV filter substances lying on the skin and the water.

A disadvantage here is that, although diffusion of the filter substances into the water can be delayed, it cannot be prevented completely. Such products can thus lose their protective effect considerably during prolonged bathing.

The object of the present invention was thus to overcome at least some, if not all, of these disadvantages.

It was therefore surprising and unforeseeable to the expert that sun protection active ingredient combinations comprising

(a) one or more UV filter substances which bear one or more sulphonic acid groups or sulphonate groups on their molecular backbone and

(b) one or more surface-active substances, selected from the group of substances of the general structural formula

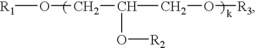

where

k is from 1 to 8,

R₁, R₂ and R₃, independently of one another, are selected from the group consisting of:

H, although in this case at least one of the radicals R₁, R₂ and R₃ must not be H,

branched or unbranched, saturated or unsaturated alkyl radicals,

branched or unbranched, saturated or unsaturated acyl radicals,

the acids on which these acyl radicals are based being selected from the group of

branched or unbranched, saturated or unsaturated alkanecarboxylic acids having from 8 to 24 carbon atoms, in which up to 3 aliphatic hydrogen atoms can be substituted by hydroxyl groups, and/or

polyester radicals of the general structure

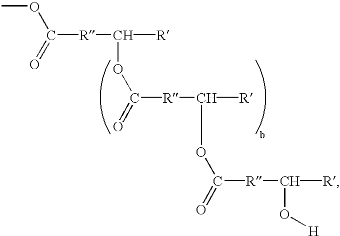

where R′ is selected from the group of branched and unbranched alkyl groups having from 1 to 20 carbon atoms, and R′ is selected from the group of branched and unbranched alkylene groups having from 1 to 20 carbon atoms, and b is from 0 to 200,

overcome the disadvantages of the prior art.

According to the invention, particularly advantageous sun protection active ingredient combinations comprise

(a) one or more UV filter substances which bear one or more sulphonic acid groups or sulphonate groups on their molecular backbone and

(b) one or more surface-active substances, selected from the group of substances of the general structural formula

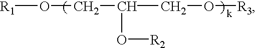

where

k is from 1 to 8,

R₁, R₂ and R₃, independently of one another, are selected from the group consisting of:

H, although in this case at least one of the radicals R₁, R₂ and R₃ must not be H,

branched or unbranched, saturated or unsaturated alkyl radicals,

branched or unbranched, saturated or unsaturated acyl radicals,

the acids on which these acyl radicals are based being selected from the group of

branched or unbranched, saturated or unsaturated alkanecarboxylic acids having from 8 to 24 carbon atoms, in which up to 3 aliphatic hydrogen atoms can be substituted by hydroxyl groups, and/or

polyester radicals of the general structure

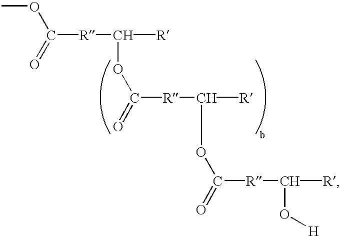

where R′ is selected from the group of branched and unbranched alkyl groups having from 1 to 20 carbon atoms, and R″ is selected from the group of branched and unbranched alkylene groups having from 1 to 20 carbon atoms, and b is from 0 to 200,

and

(c) one or more cosmetically or pharmaceutically acceptable inorganic pigments which have preferably been superficially hydrophobed.

A further advantagous embodiment of the present invention is the use of

(a) one or more cosmetically or pharmaceutically acceptable inorganic pigments which have preferably been superficially hydrophobed

for achieving or increasing the water resistance of cosmetic or dermatological sunscreen formulations which are present in the form of O/W emulsions or W/O emulsions,

(b) where the superficially hydrophobed inorganic pigments are incorporated into the oil phase of the O/W emulsions or W/O emulsions, and

(c) where, if desired, hydrophilic inorganic pigments are incorporated into the water phase of the O/W emulsions or W/O emulsions, and which comprise

(d) one or more UV filter substances which bear one or more sulphonic acid groups or sulphonate groups on their molecular backbone, and

(e) one or more surface-active substances, selected from the group of substances of the general structural formula

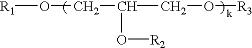

where

k is from 1 to 8,

R₁, R₂ and R₃, independently of one another, are selected from the group consisting of:

H, although in this case at least one of the radicals R₁, R₂ and R₃ must not be H,

branched or unbranched, saturated or unsaturated alkyl radicals,

branched or unbranched, saturated or unsaturated acyl radicals,

the acids on which these acyl radicals are based being selected from the group of

branched or unbranched, saturated or unsaturated alkanecarboxylic acids having from 8 to 24 carbon atoms, in which up to 3 aliphatic hydrogen atoms can be substituted by hydroxyl groups, and/or polyester radicals of the general structure

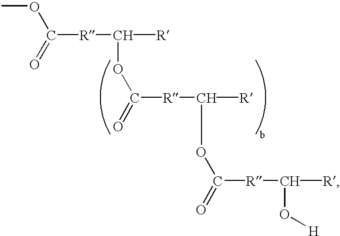

where R′ is selected from the group of branched and unbranched alkyl groups having from 1 to 20 carbon atoms, and R″ is selected from the group of branched and unbranched alkylene groups having from 1 to 20 carbon atoms, and b is from 0 to 200.

Another advantageous embodiment of the present invention is the use of

(a) one or more surface-active substances, selected from the group of substances of the general structural formula

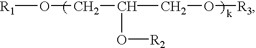

where

k is from 1 to 8,

R₁, R₂ and R₃, independently of one another, are selected from the group consisting of:

H, although in this case at least one of the radicals R₁, R₂ and R₃ must not be H, branched or unbranched, saturated or unsaturated alkyl radicals,

branched or unbranched, saturated or unsaturated acyl radicals,

the acids on which these acyl radicals are based being selected from the group of

branched or unbranched, saturated or unsaturated alkanecarboxylic acids having from 8 to 24 carbon atoms, in which up to 3 aliphatic hydrogen atoms can be substituted by hydroxyl groups, and/or

polyester radicals of the general structure

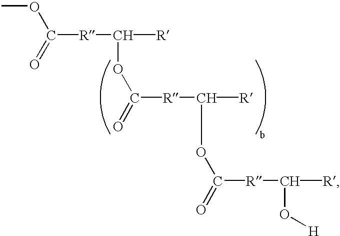

where R′ is selected from the group of branched and unbranched alkyl groups having from 1 to 20 carbon atoms, and R″ is selected from the group of branched and unbranched alkylene groups having from 1 to 20 carbon atoms, and b is from 0 to 200,

for achieving or increasing the water resistance of cosmetic or dermatological sunscreen formulations, which are present in the form of O/W emulsions or W/O emulsions, which comprise

(b) one or more UV filter substances which bear one or more sulphonic acid groups or sulphonate groups on their molecular backbone, and

which optionally further comprise

(c) one or more cosmetically or pharmaceutically acceptable inorganic pigments which are superficially hydrophobed, and which are incorporated into the oil phase of the O/W emulsions or W/O emulsions, and

(d) where any other hydrophilic inorganic pigments present are incorporated into the water phase of the O/W emulsions or W/O emulsions.

Preparations according to the invention and the described inventive uses overcome the disadvantages described for the prior art in a surprising manner. Higher sun protection factors can be achieved according to the invention than could have been assumed from the prior art.

Furthermore, it could not be foreseen from the prior art that water-resistant preparations are obtainable, according to the invention, which can achieve a considerably higher water resistance than preparations of the prior art, meaning that high sun protection factors can still be achieved even, for example, after bathing.

In the substances of the general structural formula

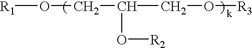

R₁, R₂ and R₃ can advantageously represent hydrogen atoms, but are also advantageously selected from the group consisting of methyl, ethyl, propyl and isopropyl, myristoyl, palmitoyl, stearoyl and eicosoyl, and from the group which is distinguished by the chemical structures

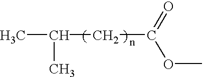

where n is from 10 to 20, the isostearoyl radical being preferred, and

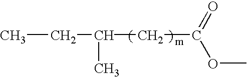

where m is from 9 to 19.

k is preferably from 1-5, particularly preferably from 2 to 4.

Sun protection active ingredient combinations according to the invention which have proved particularly advantageous are those which are monoglyceryl esters, diglyceryl esters, triglyceryl esters and are tetraglyceryl esters and monoesters of isostearic acid, tetraglyceryl monoisostearate being particularly preferred, which in analogy to the CTFA nomenclature is also called polyglyceryl-4 isostearate.

Isostearic esters of this kind are obtainable, for example, under the product name “Isolan GI 34” from Henkel Goldschmidt Chemical Corp.

Other sun protection active ingredient combinations according to the invention which have proved particularly advantageous are those which are monoglyceryl esters, diglyceryl esters, triglyceryl esters and tetraglyceryl esters and are diesters of isostearic acid, triglyceryl diisostearate being particularly preferred, which in analogy to the CTFA nomenclature is also called polyglyceryl-3 diisostearate.

Such isostearates can, for example, be obtained under the product name “Lameform TGI” from Henkel KGaA.

Other sun protection active ingredient combinations according to the invention which have proved particularly advantageous are those which are monoglyceryl esters, diglyceryl esters, triglyceryl esters and tetraglyceryl esters and comprise mixtures of monoesters and diesters of isostearic acid, approximately equimolar mixtures being particularly preferred, such as, for example, diglyceryl sesquiisostearate, which in analogy to the CTFA nomenclature is also called polyglyceryl-2 sesquiisostearate.

Such isostearates can, for example, be obtained under the product name “Hostacerin DGI” from Hoechst AG.

Sun protection active ingredient combinations according to the invention which have proved particularly advantageous are those whose polyester radicals are derived from hydroxystearic acid, “polyglyceryl-2 polyhydroxystearate” being particularly advantageous, which is assigned the registry numbers 156531-21-4 and 144470-58-6 in Chemical Abstracts, and which can, for example, be obtained under the trade name DEHYMULSO® PGPH from Henkel KGaA.

According to the invention, such surface-active substances can be present in concentrations of from 0.005 to 50% by weight, based on the total weight of the preparations. Concentrations of from 0.5-10% by weight, in particular from 1.0-5% by weight, are preferred.

Advantageous sulphonated UV filter substances in the context of the present invention are in particular:

2-phenylbenzimidazole-5-sulphonic acid and its salts, for example the sodium, potassium or its triethanolammonium salt

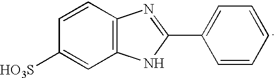

sulphonic acid derivatives of benzophenones, preferably 2-hydroxy-4-methoxybenzophenone-5-sulphonic acid and its salts, for example the corresponding sodium, potassium or triethanolammonium salt:

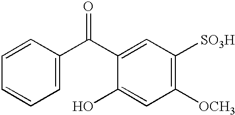

sulphonic acid derivatives of 3-benzylidenecamphor, such as, for example, 4-(2-oxo-3-bornylidenemethyl)benzenesulphonic acid, and its salts, for example the corresponding sodium, potassium or triethanolammonium salt:

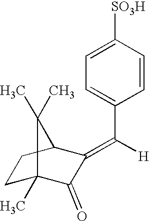

2-methyl-5-(2-oxo-3-bornylidenemethyl)benzenesulphonic acid and its salts, for example the corresponding sodium, potassium or triethanolammonium salt:

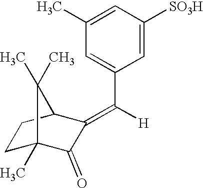

1,4-di(2-oxo-10-sulpho-3-bornylidenemethyl)benzene and its salts (the corresponding 10-sulphato compounds, for example the corresponding sodium, potassium or triethanolammonium salt), also called benzene-1,4-di(2-oxo-3-bornylidenemethyl-10-sulphonic acid):

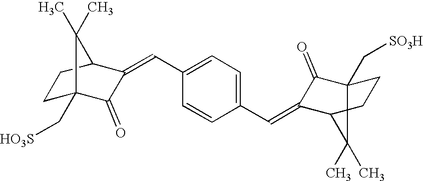

Cosmetic and dermatological preparations according to the invention comprise inorganic pigments based on metal oxides and/or other metal compounds which are sparingly soluble or insoluble in water, in particular the oxides of titanium (Tio₂), zinc (ZnO), iron (e.g. Fe₂O₃), zirconium (ZrO₂), silicon (SiO₂), manganese (e.g. MnO), aluminium (Al₂O₃), cerium (e.g. Ce₂O₃), mixed oxides of the corresponding metals and mixtures of such oxides. Particular preference is given to pigments based on TiO₂.

According to the invention, the inorganic pigments are present in hydrophobic form, i.e. they have been rendered superficially water-repellent. This surface treatment may comprise providing the pigments with a thin hydrophobic layer by processes known per se.

Such a process comprises, for example, producing the hydrophobic surface layer by a reaction in accordance with

n TiO₂+m(RO)₃Si-R′−>n TiO₂(surface).

n and m are stoichiometric parameters to be used as desired, and R and R′ are the desired organic radicals. Hydrophobed pigments prepared, for example, as in DE-A 33 14 742 are preferred.

Advantageous TiO₂ pigments are obtainable, for example, under the trade names T 805 from Degussa or MT 100 T from Tayca or M 160 from Kemira.

Any additional water-dispersible (i.e. hydrophilic) inorganic micropigments, if desired, may, for example, be those products which are obtainable under the trade name Tioveil® from Tioxide.

The total amount of inorganic pigments, in particular hydrophobic inorganic micropigments, in the finished cosmetic or dermatological preparations is advantageously chosen from the range of from 0.1-30% by weight, preferably from 0.1-10.0, in particular from 0.5-6.0% by weight, based on the total weight of the preparations.

In the cosmetic or dermatological preparations according to the invention, furthermore, the more sparingly soluble components also have a better solubility than in the preparations of the prior art, even if two or more such components are present.

Furthermore, according to the invention, the agglomeration of inorganic pigment particles (which of course are present in dispersed and not dissolved form) with the consequences of “whitening”, oil loss or emulsion breakdown, can be prevented, even if one or more components which are more sparingly soluble are also present.

Moreover, it is possible, according to the invention, to obtain sunscreen preparations which have a higher stability, in particular stability to decomposition under the influence of light, especially UV light, than could have been expected from the prior art. In particular, the stability of 4-(tert-butyl)-4′-methoxy-dibenzoylmethane is increased drastically.

Moreover, it is possible, according to the invention, to obtain preparations which are well tolerated by the skin, making distribution of valuable ingredients particularly easy.

The total amount of water-soluble UV filter substance(s) in the finished cosmetic or dermatological preparations is advantageously chosen from the range of from 0.1-10.0% by weight, preferably from 0.5-6.0% by weight, based on the total weight of the preparations.

The total amount of 2-phenylbenzimidazole-5-sulphonic acid (if it is this substance which is to be used as sulphonated UV filter substance in the context of the present invention) or salts thereof in the finished cosmetic or dermatological preparations is advantageously chosen from the range of from 0.1-10.0% by weight, preferably from 0.5-6.0% by weight, based on the total weight of the preparations.

The total amount of 2-hydroxy-4-methoxybenzophenone-5-sulphonic acid (if it is this substance which is to be used as sulphonated UW filter substance in the context of the present invention) or salts thereof in the finished cosmetic or dermatological preparations is advantageously chosen from the range of from 0.1-10.0% by weight, preferably from 0.5-6.0% by weight, based on the total weight of the preparations.

The total amount of 4-(2-oxo-3-bornylidenemethyl)benzenesulphonic acid (if it is this substance which is to be used as sulphonated UV filter substance in the context of the present invention) or salts thereof in the finished cosmetic or dermatological preparations is advantageously chosen from the range of from 0.1-10.0% by weight, preferably from 0.5-6.0% by weight, based on the total weight of the preparations.

The total amount of 2-methyl-5-(2-oxo-3-bornylidenemethyl)benzenesulphonic acid (if it is this substance which is to be used as sulphonated UV filter substance in the context of the present invention) or salts thereof in the finished cosmetic or dermatological preparations is advantageously chosen from the range of from 0.1-10.0% by weight, preferably from 0.5-6.0% by weight, based on the total weight of the preparations.

The total amount of benzene-1,4-di(2-oxo-3-bornylidenemethyl-10-sulphonic acid) (if it is this substance which is to be used as sulphonated UV filter substance in the context of the present invention) or salts thereof in the finished cosmetic or dermatological preparations is advantageously chosen from the range of from 0.1-10.0% by weight, preferably from 0.5-6.0% by weight, based on the total weight of the preparations.

The total amount of tris(2-ethylhexyl) 4,4′,4″-(1,3,5-triazine-2,4,6-triyltriimino)trisbenzoate (as additional UV filter substance which is optionally to be used per se) in the finished cosmetic or dermatological preparations is advantageously chosen from the range of from 0.1-10.0% by weight, preferably from 0.5-6.0% by weight, based on the total weight of the preparations.

The total amount of 4-(tert-butyl)-4′-methoxydibenzoylmethane (as additional UV filter substance which is optionally to be used per se) in the finished cosmetic or dermatological preparations is advantageously chosen from the range of from 0.1-10.0% by weight, preferably from 0.5-6.0% by weight, based on the total weight of the preparations.

The total amount of 4-methylbenzylidenecamphor (as additional UV filter substance which is optionally to be used per se) in the finished cosmetic or dermatological preparations is advantageously chosen from the range of from 0.1-10.0% by weight, preferably from 0.5-6.0% by weight, based on the total weight of the preparations.

The total amount of 2-ethylhexyl p-methoxy-cinnamate (as additional UV filter substance which is optionally to be used per se) in the finished cosmetic or dermatological preparations is advantageously chosen from the range of from 0.1-15.0% by weight, preferably from 0.5-7.5% by weight, based on the total weight of the preparations.

The total amount of ethylhexyl 2-cyano-3,3-diphenylacrylate (as additional UV filter substance which is optionally to be used per se) in the finished cosmetic or dermatological preparations is advantageously chosen from the range of from 0.1-15.0% by weight, preferably from 0.5-10.0% by weight, based on the total weight of the preparations.

Furthermore it is advantageous to combine the active ingredient combinations according to the invention with further UVA and/or UVB filters.

It may furthermore be advantageous, where appropriate, to combine the active ingredient combinations according to the invention with further UVA and/or UVB filters, for example certain salicylic acid derivatives, such as

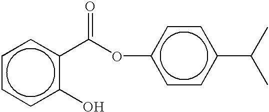

(4-isopropybenzyl salicylate),

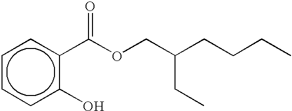

(2-ethylhexyl salicylate, octyl salicylate),

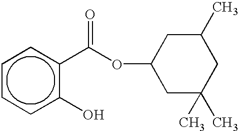

(homomenthyl salicylate).

The total amount of one or more salicylic acid derivatives in the finished cosmetic or dermatological preparations is advantageously chosen from the range of from 0.1-15.0% by weight, preferably from 0.5-8.0% by weight, based on the total weight of the preparations. If ethylhexyl salicylate is chosen, it is advantageous to choose the total amount thereof from the range of from 0.1-5.0% by weight, preferably from 0.5-2.5% by weight. If homomenthyl salicylate is chosen, it is advantageous to choose the total amount thereof from the range of 0.1-10.0% by weight, preferably 0.5-5.0% by weight.

It may also be advantageous to combine the combinations according to the invention with UVA filters which have usually been present in cosmetic preparations to date. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. These combinations, and preparations which comprise these combinations, are also provided by the invention. The amounts used for the UVB combination can be used.

It is advantageous according to the invention to use in addition to the combinations according to the invention, further UVA filters and/or UVB filters, the total amount of filter substances being, for example, from 0.1% by weight to 30% by weight, preferably from 0.5 to 10% by weight, in particular from 1 to 6% by weight, based on the total weight of the preparations, in order to provide cosmetic preparations which protect the skin from the entire range of ultraviolet radiation. They can also serve as sunscreen products.

Advantageous oil-soluble UVB filter substances are, for example:

3-benzylidenecamphor derivatives, preferably 3-(4-methylbenzylidene) camphor and 3-benzylidenecamphor;

4-aminobenzoic acid derivatives, preferably 2-ethylhexyl 4-(dimethylamino)benzoate and amyl 4-(dimethylamino)benzoate;

esters of cinnamic acid, preferably 2-ethylhexyl 4-methoxycinnamate and isopentyl 4-methoxycinnamate;

derivatives of benzophenone, preferably 2-hydroxy-4-methoxybenzophenone, 2-hydroxy-4-methoxy-4′-methylbenzophenone and 2,2′-dihydroxy-4-methoxybenzophenone;

esters of benzalmalonic acid, preferably di(2-ethylhexyl) 4-methoxybenzalmalonate;

2,4,6-trianilino-(p-carbo-2′-ethyl-1′-hexyloxy)-1,3,5-triazine.

The list of UVB filters mentioned which can be used in combination with the active ingredient combinations according to the invention is of course not intended to be limiting.

As mentioned above, the present invention allows a comparable or even higher sunscreen filter action to be achieved than was allowed to date by the prior art, for a lower total concentration of UV filter substances. It has also proved to be particularly advantageous to introduce an additional content of cosmetically or pharmaceutically acceptable electrolytes. Over wide concentration ranges, it is possible for the concentration of the UV filter substance or substances to be reduced by the same or certainly at least a comparable amount as that with which the preparation is topped up, as it were, with one or more electrolytes. The lower limit at which this behaviour manifests itself in a manner relevant to the consumer has as a rule proved to be a total content of about 0.5% by weight of UV filter substances.

The preparations according to the invention therefore advantageously comprise electrolytes, in particular one or more salts with the following anions: chlorides, and furthermore inorganic oxo element anions, and of these in particular sulphates, carbonates, phosphates, borates and aluminates. Electrolytes based on organic anions can also advantageously be used, for example lactates, acetates, benzoates, propionates, tartrates, citrates and many others. Comparable effects can also be achieved by ethylenediaminetetraacetic acid and salts thereof.

Cations of the salts which are preferably used are ammonium, alkylammonium, alkali metal, alkaline earth metal, magnesium, iron and zinc ions. It requires no mention per se that only physiologically acceptable electrolytes should be used in cosmetics. Particular preference is given to potassium chloride, sodium chloride, magnesium sulphate, zinc sulphate and mixtures thereof. Salt mixtures such as occur in the natural salt from the Dead Sea are also advantageous.

The concentration of the electrolyte(s) should be from about 0.1-10.0% by weight, particularly advantageously from about 0.3-8.0% by weight, based on the total weight of the preparation.

The cosmetic and/or dermatological sunscreen preparations according to the invention can have the customary composition and be used for cosmetic and/or dermatological sun protection, and also for treatment, care and cleansing of the skin and/or hair and as a make-up product in decorative cosmetics.

For use, the cosmetic and dermatological preparations according to the invention are applied to the skin and/or hair in an adequate amount in the manner customary for cosmetics.

Those cosmetic and dermatological preparations which are in the form of a sunscreen product are particularly preferred. These can additionally comprise at least one further UVA filter and/or at least one further UVB filter.

The cosmetic and dermatological preparations according to the invention can comprise cosmetic auxiliaries such as are usually used in such preparations, for example preservatives, bactericides, perfumes, dyes, pigments which have a colouring action, thickeners, humidifying and/or humectant substances, fats, oils, waxes or other customary constituents of a cosmetic or dermatological formulation, such as alcohols, polyols, polymers, foam stabilizers, electrolytes, organic solvents, further emulsifiers or silicone derivatives.

An additional content of antioxidants is in general preferred. Favourable antioxidants which can be used according to the invention are all the antioxidants which are suitable or customary for cosmetic and/or dermatological applications.

The antioxidants are advantageously chosen from the group consisting of amino acids (for example glycine, histidine, tyrosine and tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene and lycopene) and derivatives thereof, chlorogenic acid and derivatives thereof, liponic acid and derivatives thereof (for example dihydroliponic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulphoximine compounds (for example buthionine sulphoximines, homocysteine sulphoximine, buthionine sulphones and penta-, hexa- and heptathionine sulphoximine) in very low tolerated dosages (for example pmol to μmol/kg), and furthermore (metal) chelators (for example α-hydroxy fatty acids, palmitic acid, phytic acid and lactoferrin), α-hydroxy acids (for example citric acid, lactic acid and malic acid), humic acid, bile acid, bile extracts, bilirubin, biliverdin, EDTA, EGTA and derivatives thereof, unsaturated fatty acids and derivatives thereof (for example γ-linolenic acid, linoleic acid and oleic acid), folic acid and derivatives thereof, ubiquinone and ubiquinol and derivatives thereof, vitamin C and derivatives (for example ascorbyl palmitate, Mg ascorbyl phosphate and ascorbyl acetate), tocopherols and derivatives (for example vitamin E acetate), vitamin A and derivatives (vitamin A palmitate) and coniferyl benzoate of benzoin resin, rutic acid and derivatives thereof, α-glycosylrutin, ferulic acid, furfurylideneglucitol, carnosine, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (for example ZnO and ZnSO₄), selenium and derivatives thereof (for example selenium-methionine), stilbene and derivatives thereof (for example stilbene oxide and trans-stilbene oxide) and the derivatives of these active ingredients mentioned which are suitable according to the invention (salts, esters, ethers, sugars, nucleotides, nucleosides, peptides and lipids).

The amount of the abovementioned antioxidants (one or more compounds) in the preparations is preferably from 0.001 to 30% by weight, particularly preferably from 0.05-20% by weight, in particular from 1-10% by weight, based on the total weight of the preparation.

If vitamin E and/or derivatives thereof are the antioxidant(s), it is advantageous to choose the particular concentrations thereof from the range of from 0.001-10% by weight, based on the total weight of the preparation.

If vitamin A or vitamin A derivatives or carotenes or derivatives thereof are the antioxidant(s), it is advantageous to choose the particular concentrations thereof from the range of from 0.001-10% by weight, based on the total weight of the preparation.

The oil phase of the preparations according to the invention is advantageously chosen from the group consisting of esters of saturated and/or unsaturated, branched and/or unbranched alkanecarboxylic acids having a chain length of from 3 to 30 C atoms and saturated and/or unsaturated, branched and/or unbranched alcohols having a chain length of from 3 to 30 C atoms, or from the group consisting of esters of aromatic carboxylic acids and saturated and/or unsaturated branched and/or unbranched alcohols having a chain length of from 3 to 30 C atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and naturally occurring mixtures of such esters, for example jojoba oil.

Furthermore, the oil phase can advantageously be chosen from the group consisting of branched and unbranched hydrocarbons and waxes, silicone oils, dialkyl ethers, from the group consisting of saturated or unsaturated, branched or unbranched alcohols, and fatty acid triglycerides, especially the triglyceryl esters of saturated and/or unsaturated, branched and/or unbranched alkanecarboxylic acids having a chain length of from 8 to 24, in particular from 12-18 C atoms. The fatty acid triglycerides can advantageously be chosen, for example, from the group consisting of synthetic, semi-synthetic and naturally occurring oils, for example olive oil, sunflower oil, soya oil, groundnut oil, rapeseed oil, almond oil, palm oil, coconut oil, palm kernel oil and many others of this type.

Any desired mixtures of such oil and wax components are also advantageously to be used in the context of the present invention.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/ capric triglyceride and dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous.

Of the hydrocarbons, paraffin oil, squalane and squalene are advantageously to be used in the context of the present invention.

Furthermore, the oil phase can advantageously contain cyclic or linear silicone oils or consist entirely of such oils, although it is preferable to use an additional content of other oil phase components in addition to the silicone oil or silicone oils.

Cyclomethicone (octamethylcyclotetrasiloxane) is advantageously used as a silicone oil to be used according to the invention. However, other silicone oils are also advantageously to be used in the context of the present invention, for example hexamethylcyclotrisiloxane, polydimethylsiloxane and poly(methylphenylsiloxane).

Mixtures of cyclomethicone and isotridecyl isononanoate and of cyclomethicone and 2-ethylhexyl isostearate are furthermore particularly advantageous.

The content of the oil phase is advantageously between 1 and 50% by weight, based on the total weight of the preparations, preferably 2.5-30% by weight, particularly preferably 5-15% by weight.

The aqueous phase of the preparations according to the invention optionally advantageously comprises alcohols, diols or polyols of low C number, as well as ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low C number, for example ethanol, isopropanol, 1,2-propanediol and glycerol, and, in particular, one or more thickeners, which can advantageously be chosen from the group consisting of silicon dioxide, aluminium silicates, polysaccharides and derivatives thereof, for example hyaluronic acid, xanthan gum and hydroxypropylmethylcellulose, particularly advantageously from the group consisting of polyacrylates, preferably a polyacrylate from the group consisting of the so-called Carbopols, for example Carbopols of types 980, 981, 1382, 2984 and 5984, in each case individually or in combination.

The following examples are intended to illustrate the present invention without limiting it. Unless stated otherwise, all the amounts, contents and percentage contents are based on the weight and the total amount or on the total weight of the preparations.

| | % by weight |
|---|---|
| Example 1 | |
| Polyglyceryl-4 isostearate | 5.00 |
| Caprylic/capric triglycerides | 5.00 |
| Octyldodecanol | 5.00 |
| Dicaprylyl ether | 2.00 |
| Phenylbenzimidazolesulphonic acid | 4.00 |
| TiO₂ (hydrophobic) | 3.00 |
| Glycerin | 3.00 |
| Tocopheryl acetate | 1.00 |
| NaOH | q.s. |
| Perfume, preservative | q.s. |
| Water | to 100.00 |
| Example 2 | |
| Polyglyceryl-4 isostearate | 5.00 |
| Caprylic/capric triglycerides | 1.67 |
| Octyldodecanol | 1.67 |
| Dicaprylyl ether | 1.67 |
| Phenylbenzimidazolesulphnoic acid | 4.00 |
| TiO₂ (hydrophobic) | 5.00 |
| TiO₂ (hydrophilic) | 5.00 |
| Butylene glycol | 3.00 |
| Tocopheryl acetate | 1.00 |
| NaOH | q.s. |
| Perfume, preservative | q.s. |
| Water | to 100.00 |
| Example 3 | |
| Polyglyceryl-4 isostearate | 3.00 |
| Caprylic/capric triglycerides | 5.00 |
| Octyldecanol | 5.00 |
| Dicaprylyl ether | 1.67 |
| Benzene-1,4-di(2-oxo-3-bornylidene- | 4.00 |
| methyl-10-sulphnoic acid) | |
| TiO₂ (hydrophobic) | 5.00 |
| Glyceryl monostearate | 2.00 |
| Glycerin | 3.00 |
| Tocopheryl acetate | 1.00 |
| NaOH | q.s. |
| Perfume, preservative | q.s. |
| Water | to 100.00 |
| Example 4 | |
| Polyglyceryl-4 isostearate | 5.00 |
| Caprylic/capric triglycerides | 1.67 |
| Octyldodecanol | 1.67 |
| C₁₂₋₁₅-Alkyl benzoates | 5.00 |
| Phenylbenzimidazolesulphonic acid | 2.00 |
| TiO₂ (hydrophobic) | 2.50 |
| Tris[anilino(p-carbo-2′-ethyl- | 3.00 |
| 1′-hexyloxy)]triazine | |
| 4-(tert-Butyl)-4′-methoxydibenzoyl- | 2.00 |
| methane | |
| Butylene glycol | 3.00 |
| 4-Methylbenzylidenecamphor | 2.00 |
| Tocopheryl acetate | 1.00 |
| Sorbitan monostearate | 2.00 |
| NaOH | q.s. |
| Perfume, preservative | q.s. |
| Water | to 100.00 |
| Example 5 | |
| Polyglyceryl-3 diisostearate | 5.00 |
| Caprylic/capric triglycerides | 5.00 |
| Octyldodecanol | 5.00 |
| Dicaprylyl ether | 2.00 |
| Phenylbenzimidazolesulphnoic acid | 4.00 |
| TiO₂ (hydrophobic) | 3.00 |
| Glycerin | 3.00 |
| Tocophenyl acetate | 1.00 |
| NaOH | q.s. |
| Perfume, preservative | q.s. |
| Water | to 100.00 |
| Example 6 | |
| Polyglyceryl-3 diisostearate | 5.00 |
| Caprylic/capric triglycerides | 1.67 |
| Octyldecanol | 1.67 |
| Dicaprylyl ether | 1.67 |
| Phenylbenzimidazolesulphonic acid | 4.00 |
| TiO₂ (hydrophobic) | 5.00 |
| TiO₂ (hydrophilic) | 5.00 |
| Butylene glycol | 3.00 |
| Tocopheryl acetate | 1.00 |
| NaOH | q.s. |
| Perfume, preservative | q.s. |
| Water | to 100.00 |
| Example 7 | |
| Polyglyceryl-4 diisostearate | 3.00 |
| Caprylic/capric triglycerides | 5.00 |
| Octyldodecanol | 5.00 |
| Dicaprylyl ether | 1.67 |
| Benzene-1,4-di(2-oxo-3-bornylidene- | 4.00 |
| methyl-10-sulphonic acid) | |
| TiO₂ (hydrophobic) | 5.00 |
| Glycerin monostearate | 2.00 |
| Glycerin | 3.00 |
| Tocopheryl acetate | 1.00 |
| NaOH | q.s. |
| Perfume, preservative | q.s. |
| Water | to 100.00 |
| Example 8 | |
| Polyglyceryl-3 diisostearate | 5.00 |
| Caprylic/capric triglycerides | 1.67 |
| Octyldodecanol | 1.67 |
| C₁₂₋₁₅-Alkyl benzoates | 5.00 |
| Phenylbenzimidazolesulphonic acid | 2.00 |
| TiO₂ (hydrophobic) | 2.50 |
| Tris[anilino(p-carbo-2′-ethyl- | 3.00 |
| 1′-hexyloxy)]triazine | |
| 4-(tert-Butyl)-4′-methoxydibenzoyl- | 2.00 |
| methane | |
| Butylene glycol | 3.00 |
| 4-Methylbenzylidenecamphor | 2.00 |
| Tocopheryl acetate | 1.00 |
| Sorbitan monostearate | 2.00 |
| NaOH | q.s. |
| Perfume, preservative | q.s. |
| Water | to 100.00 |
| Example 9 | |
| Polyglyceryl-2 sesquiisostearate | 5.00 |
| Caprylic/capric triglycerides | 5.00 |
| Octyldodecanol | 5.00 |
| Dicaprylyl ether | 2.00 |
| Phenylbenzimidazolesulphonic acid | 4.00 |
| TiO₂ (hydrophobic) | 3.00 |
| Glycerin | 3.00 |
| Tocopheryl acetate | 1.00 |
| NaOH | q.s. |
| Perfume, preservative | q.s. |
| Water | to 100.00 |
| Example 10 | |
| Polyglyceryl-2 sesquiisostearate | 5.00 |
| Caprylic/capric triglycerides | 1.67 |
| Octyldodecanol | 1.67 |
| Dicaprylyl ether | 1.67 |
| Phenylbenzimidazolesulphonic acid | 4.00 |
| TiO₂ (hydrophobic) | 5.00 |
| TiO₂ (hydrophilic) | 5.00 |
| Butylene glycol | 3.00 |
| Tocopheryl acetate | 1.00 |
| NaOH | q.s. |
| Perfume, preservative | q.s. |
| Water | to 100.00 |
| Example 11 | |
| Polyglyceryl-2 sesquiisostearate | 3.00 |
| Caprylic/capric triglycerides | 5.00 |
| Octyldodecanol | 5.00 |
| Dicaprylyl ether | 1.67 |
| Benzene-1,4-di(2-oxo-3-bornylidene- | 4.00 |
| methyl-10-sulphnoic acid) | |
| TiO₂ (hydrophobic) | 5.00 |
| Glyceryl monostearate | 2.00 |
| Glycerin | 3.00 |
| Tocopheryl acetate | 1.00 |
| NaOH | q.s. |
| Perfume, preservative | q.s. |
| Water | to 100.00 |
| Example 12 | |
| Polyglyceryl-2 sesquiisostearate | 5.00 |
| Caprylic/capric triglycerides | 1.67 |
| Octyldodecanol | 1.67 |
| C₁₂₋₁₅-Alkyl benzoates | 5.00 |
| Phenylbenzimidazolesulphonic acid | 2.00 |
| TiO₂ (hydrophobic) | 2.50 |
| Tris[anilino(p-carbo-2′-ethyl- | 3.00 |
| 1′-hexyloxy)]triazine | |
| 4-(tert-Butyl)-4′-methoxydibenzoyl- | 2.00 |
| methane | |
| Butylene glycol | 3.00 |
| 4-Methylbenzylidenecamphor | 2.00 |
| Tocopheryl acetate | 1.00 |
| Sorbitan monostearate | 2.00 |
| NaOH | q.s. |
| Perfume, preservative | q.s. |
| Water | to 100.00 |
| Example 13 | |
| Polyglyceryl-2 polyhydroxystearate | 5.00 |
| Caprylic/capric triglycerides | 5.00 |
| Octyldodecanol | 5.00 |
| Dicaprylyl ether | 2.00 |
| Phenylbenzimidazolesulphonic acid | 4.00 |
| TiO₂ (hydrophobic) | 3.00 |
| Glycerin | 3.00 |
| Tocopheryl acetate | 1.00 |
| NaOH | q.s. |
| Perfume, preservative | q.s. |
| Water | to 100.00 |
| Example 14 | |
| Polyglyceryl-2 polyhydroxystearate | 5.00 |
| Caprylic/capric triglycerides | 1.67 |
| Octyldodecanol | 1.67 |
| Dicaprylyl ether | 1.67 |
| Phenylbenzimidazolesulphonic acid | 4.00 |
| TiO₂ (hydrophobic) | 5.00 |
| TiO₂ (hydrophilic) | 5.00 |
| Butylene glycol | 3.00 |
| Tocopheryl acetate | 1.00 |
| NaOH | q.s. |
| Perfume, preservative | q.s. |
| Water | to 100.00 |
| Example 15 | |
| Polyglyceryl-2 polyhydroxystearate | 3.00 |
| Caprylic/capric triglycerides | 5.00 |
| Octyldodecanol | 5.00 |
| Dicaprylyl ether | 1.67 |
| Benzene-1,4-di(2-oxo-3-bornylidene- | 4.00 |
| methyl-10-sulphnoic acid | |
| TiO₂ (hydrophobic) | 5.00 |
| Glyceryl monostearate | 2.00 |
| Glycerin | 3.00 |
| Tocopheryl acetate | 1.00 |
| NaOH | q.s. |
| Perfume, preservative | q.s. |
| Water | to 100.00 |
| Example 16 | |
| Polyglyceryl-2 polyhydroxystearate | 5.00 |
| Caprylic/capric triglycerides | 1.67 |
| Octyldodecanol | 1.67 |
| C₁₂₋₁₅-Alkyl benzoates | 5.00 |
| Phenylbenzimidazolesulphonic acid | 2.00 |
| TiO₂ (hydrophobic) | 2.50 |
| Tris[anilino(p-carbo-2′-ethyl- | 3.00 |
| 1′-hexyloxy)]triazine | |
| 4-(tert-Butyl)-4′-methoxydibenzoyl- | 2.00 |
| methane | |
| Butylene glycol | 3.00 |
| 4-Methylbenzylidenecamphor | 2.00 |
| Tocopheryl acetate | 1.00 |
| Sorbitan monostearate | 2.00 |
| NaOH | q.s. |
| Perfume, preservative | q.s. |
| Water | to 100.00 |

1. Sun protection active ingredient combinations comprising
(a) one or more UV filter substances which bear one or more sulphonic acid groups or sulphonate groups on their molecular backbone and
(b) one or more surface-active substances, selected from the group of substances of the general structural formula 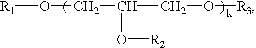
where
k is from 1 to 8,
R₁, R₂ and R₃, in dependently of one another, are selected from the group consisting of:
H, although in this case at least one of the radicals R₁, R₂ and R₃ must not be H,
branched or unbranched, saturated or unsaturated alkyl radicals,
branched or unbranched, saturated or unsaturated acyl radicals,
the acids on which these acyl radicals are based being selected from the group of
branched or unbranched, saturated or unsaturated alkanecarboxylic acids having from 8 to 24 carbon atoms, in which up to 3 aliphatic hydrogen atoms can be substituted by hydroxyl groups, and/or
polyester radicals of the general structure 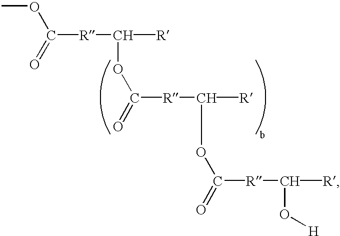
where R′ is selected from the group of branched and unbranched alkyl groups having from 1 to 20 carbon atoms, and R″ is selected from the group of branched and unbranched alkylene groups having from 1 to 20 carbon atoms, and b is from 0 to 200.

2. Sun protection active ingredient combinations according to claim 1 which also comprise one or more cosmetically or pharmaceutically acceptable inorganic pigments, which have preferably been superficially hydrophobed.

3. Use of
(a) one or more cosmetically or pharmaceutically acceptable inorganic pigments which have preferably been superficially hydrophobed
for achieving or increasing the water resistance of cosmetic or dermatological sunscreen preparations which are present in the form of O/W emulsions or W/O emulsions,
(b) where the superficially hydrophobed inorganic pigments are incorporated into the oil phase of the O/W emulsions or W/O emulsions, and
(c) where, if desired, hydrophilic inorganic pigments are incorporated into the water phase of the O/W emulsions or W/O emulsions, and
which comprise
(d) one or more UV filter substances which bear one or more sulphonic acid groups or sulphonate groups on their molecular backbone, and
(e) one or more surface-active substances, selected from the group of substances of the general structural formula 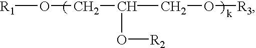
where
k is from 1 to 8,
R₁, R₂ and R₃, independently of one another, are selected from the group consisting of:
H, although in this case at least one of the radicals R₁, R₂ and R₃ must not be H,
branched or unbranched, saturated or unsaturated alkyl radicals,
branched or unbranched, saturated or unsaturated acyl radicals,
the acids on which these acyl radicals are based being selected from the group of
branched or unbranched, saturated or unsaturated alkanecarboxylic acids having from 8 to 24 carbon atoms, in which up to 3 aliphatic hydrogen atoms can be substituted by hydroxyl groups, and/or
polyester radicals of the general structure 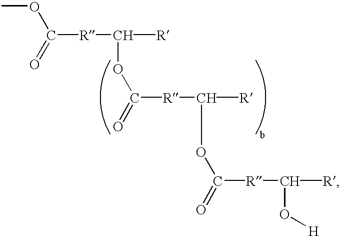
where R′ is selected from the group of branched and unbranched alkyl groups having from 1 to 20 carbon atoms, and R″ is selected from the group of branched and unbranched alkylene groups having from 1 to 20 carbon atoms, and b is from 0 to 200.

4. Use of
(a) one or more surface-active substances, selected from the group of substances of the general structural formula 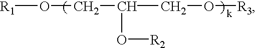
where
k is from 1 to 8,
R₁, R₂ and R₃, independently of one another, are selected from the group consisting of:
H, although in this case at least one of the radicals R₁, R₂ and R₃ must not be H,
branched or unbranched, saturated or unsaturated alkyl radicals,
branched or unbranched, saturated or unsaturated acyl radicals,
the acids on which these acyl radicals are based being selected from the group of
branched or unbranched, saturated or unsaturated alkanecarboxylic acids having from 8 to 24 carbon atoms, in which up to 3 aliphatic hydrogen atoms can be substituted by hydroxyl groups, and/or
polyester radicals of the general structure 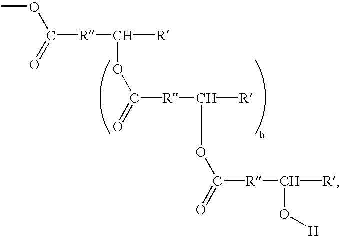
where R′ is selected from the group of branched and unbranched alkyl groups having from 1 to 20 carbon atoms, and R″ is selected from the group of branched and unbranched alkylene groups having from 1 to 20 carbon atoms, and b is from 0 to 200,
for achieving or increasing the water resistance of cosmetic or dermatological sunscreen preparations, which are present in the form of O/W emulsions or W/O emulsions, which comprise
(b) one or more UV filter substances which bear one or more sulphonic acid groups or sulphonate groups on their molecular backbone, and
which optionally further comprise
(c) one or more cosmetically or pharmaceutically acceptable inorganic pigments which are superficially hydrophobed, and which are incorporated into the oil phase of the O/W emulsions or W/O emulsions, and
(d) where any other hydrophilic inorganic pigments present are incorporated into the water phase of the O/W emulsions or W/O emulsions.

5. Preparations according to claim 1 or 2 or uses according to claim 3 or 4, characterized in that in the substances of the general structural formula 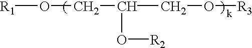
R₁, R₂ and R₃ are selected from H, methyl, ethyl, propyl, isopropyl, myristoyl, palmitoyl, stearoyl and eicosoyl groups, or from the group which is distinguished by the chemical structures 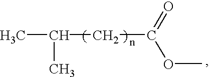
where n is from 10 to 20, the isostearyol radical being preferred, and 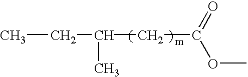
where m is from 9 to 19.

6. Preparations according to claim 1 or 2 or uses according to claim 3 or 4, characterized in that the substances of the general structural formula 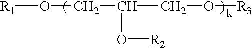
are selected from the group consisting of polyglyceryl-4 isostearate, polyglyceryl-3 diisostearate, polyglyceryl-2 sesquiisostearate and polyglyceryl-2 polyhydroxystearate.

7. Preparations according to claim 1 or 2 or uses according to claim 3 or 4, characterized in that the substances of the general structural formula 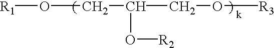
are present in concentrations of from 0.005 to 50% by weight, preferably in concentrations of from 0.5 to 10% by weight, in particular from 1.0 to 5% by weight, based on the total weight of the preparations.
